(19) United States
(12) Patent Application Publication
(10) Pub. No.: US 2026/0309693 A1
(43) Pub. Date: Oct. 8, 2026

(54) NANOPORE SEQUENCING AND PREPARATION MODULAR INSTRUMENT

(71) Applicant: Oxford Nanopore Technologies PLC, Oxford (GB)

(72) Inventor: Clive Gavin Brown, Oxford (GB)

(73) Assignee: Oxford Nanopore Technologies PLC, Oxford (GB)

(21) Appl. No.: 19/485,042

(22) PCT Filed: May 17, 2024
(86) PCT No.: PCT/GB2024/051291

§ 371 Date: Nov. 17, 2025

(30) Foreign Application Priority Data
May 18, 2023 (GB) 2307494.1

(51) Int. Cl. G01N 33/487

(52) U.S. Cl. CPC G01N 33/48721

(57) ABSTRACT

A modular instrument (1) for preparing and sensing an analyte in a prepared sample. The modular instrument includes a main body (12), an EWOD sample preparation device (2), and a nanopore sensor device (3) for sensing the analyte. The main body is electrically connectable to and separable from both the EWOD sample preparation device and the nanopore sensor device. The main body, the EWOD sample preparation device and nanopore sensor are physically connectable to and separable from each other.

### RELATED APPLICATIONS

This application is a national stage filing under 35 U.S.C. § 371 of international application number PCT/GB 2024/051291, filed May 17, 2024, which claims the benefit of United Kingdom application number GB 2307494.1, filed May 18, 2023, each of which is herein incorporated by reference in its entirety.

### FIELD OF INVENTION

The present invention relates to a modular instrument for preparing samples for, and testing samples using, nanopore sequencing.

### BACKGROUND

The preparation of samples for use with nanopore sensor devices is often complex, owing to different preparation protocols that may be required, before the prepared sample is ready to be transferred into a nanopore sensor device for analysis. Dedicated sample preparation devices, such as an electro-wetting on dielectric (EWOD) device, e.g. the VolTRAX™ device sold by Oxford Nanopore Technologies, may be used to control the interaction of the sample with multiple different reagents, before the prepared sample is ready to be transferred by the user, via pipette, to a nanopore sensor device, such as the MinION™ device, also sold by Oxford Nanopore Technologies.

It is an aim of the present invention to provide an improved system for the preparation of samples for, and testing of samples using, nanopore sequencing.

When viewed from a first aspect the invention provides a modular instrument for preparing and sensing an analyte in a prepared sample, the modular instrument comprising:
a main body;
an EWOD sample preparation device for the preparation of the prepared sample for use with a nanopore sensor device; and
a nanopore sensor device for sensing the analyte;
wherein the main body is electrically connectable to and separable from both the EWOD sample preparation device and the nanopore sensor device;
wherein the main body, the EWOD sample preparation device and nanopore sensor are physically connectable to and separable from each other; and
wherein the EWOD sample preparation device comprises:
   an interior volume for interacting a raw sample with one or more reagents;
   a sample inlet for inputting the raw sample into the interior volume;
   a plurality of reagent inlets for inputting the one or more reagents into the interior volume; and
   an outlet for outputting the prepared sample into an inlet of the nanopore sensor device for sensing of the analyte, wherein the outlet of the EWOD sample preparation device is fluidically connectable to the inlet of the nanopore device.

### SUMMARY OF THE INVENTION

The present invention provides a modular instrument for both preparing a prepared sample for testing and testing the prepared sample using a sensor device that includes a nanopore. The modular instrument includes an EWOD sample preparation device arranged to prepare a prepared sample for testing (by the nanopore sensor device) and the nanopore sensor device.

The use of the term raw sample includes both processed and unprocessed (or refined and unrefined) samples such as bodily fluids from, for example, patients or test subjects. It may also include refined or at least partially refined samples, treated samples, saturated samples, etc., that can be input into the modular instrument for analysis. In addition, the raw sample is prepared and processed in the EWOD sample preparation device to provide a prepared sample. The terms raw sample and prepared sample are used to refer to the initial and final states of the sample for analysis respectively, and the term raw sample is used to refer to all intermediate states of the sample as it is being prepared in the device.

Raw samples may include refined or unrefined biological samples (such as blood, spit, mucous, sap, etc.). Samples may also include refined or unrefined parts of sewage, soil, river water, surface swabs, etc.

The term EWOD is a well-known technique for manipulating droplets of liquid by application of an electric field. The EWOD may be an Active Matrix EWOD (AM-EWOD) having an active matrix array incorporating transistors, for example by using thin film transistors (TFTs). The device comprises upper and lower substrates defining a fluid chamber and an electrode array provided on the interior of one of the substrates wherein the electric field between the electrodes may be controlled to move, mix, separate and merge droplets disclosed by US 2015/158028, US 2014/197028, US 2014/202863 and US 2016/305906, hereby incorporated by reference in their entirety.

The EWOD sample preparation device includes an interior volume in which the sample is able to interact with one or more reagents. The EWOD sample preparation device has an inlet for inputting the raw sample into the interior volume and an outlet for outputting the prepared sample from the EWOD sample preparation device. The EWOD sample preparation device also has multiple inlets for inputting one or more reagents into the interior volume. The reagents may be used, in examples for interacting (in some instances reacting) with the raw sample, in the preparation of the raw sample within the interior volume, before the prepared sample is output from the interior volume through the outlet.

The nanopore sensor device has an inlet for receiving the prepared sample that is output from the EWOD sample preparation device. The nanopore sensor device is arranged, with the EWOD sample preparation device, such that when the nanopore sensor device is displaced relative to (i.e. inserted into, moved within and/or withdrawn from) the outlet of the EWOD sample preparation device, the nanopore sensor device is arranged to receive the prepared sample from the outlet of the EWOD sample preparation device, through the inlet of the nanopore sensor device.

It will be appreciated that by integrating the nanopore sensor device into an instrument with the EWOD sample preparation device, such that the prepared sample can be transferred directly from the EWOD sample preparation device to the nanopore sensor device, this reduces (or may eliminate) human involvement (for example, pipetting) in the process. As a result, the process of preparing and testing a prepared sample may be more reliable and efficient, as it may be less prone to errors and variation in the process. Such errors and variation may, in conventional devices, arise from the presence of bubbles or different volumes being extracted from the EWOD sample preparation device when pipetting.

In some embodiments, the EWOD sample preparation device comprises a port for (i.e. receiving or communicating with) the nanopore sensor device, into which the nanopore sensor device may be able to be inserted. This may allow the nanopore sensor device to be inserted into the port to receive the prepared sample from the outlet of the EWOD sample preparation device.

In some embodiments, the main body comprises a port for (i.e. receiving or communicating with) the nanopore sensor device, into which the nanopore sensor device may be able to be inserted. This may allow the nanopore sensor device to be inserted into the port for the prepared sample to be tested.

When the main body comprises a port for the nanopore sensor device, the nanopore sensor device may first receive the prepared sample from the EWOD sample preparation device, for example by inserting the nanopore sensor device into the (in some instances the port of the) EWOD sample preparation device. The nanopore sensor device may then be removed from the (in some instances the port of the) EWOD sample preparation device and be inserted into the port of the main body, for the prepared sample to be tested. This allows the functions of the main body and the EWOD sample preparation device to be separated, so that they can be designed and manufactured specifically for these separate functions.

Thus, preferably the port of the main body comprises an electrical connection for electrically connecting to the nanopore sensor device, and for electrically connecting the nanopore sensor device to the main body.

In some embodiments, the nanopore sensor device is left in the (in some instances the port of the) EWOD sample preparation device, after receiving the prepared sample from the EWOD sample preparation device. This helps to simplify operation of the instrument, as the nanopore sensor device may just need to be inserted into the EWOD sample preparation device, both for the prepared sample to be retrieved from the EWOD sample preparation device and for the prepared sample to be tested.

Thus, in some embodiments, the (in some instances the port of the) EWOD sample preparation device comprises an electrical connection for electrically connecting to the nanopore sensor device, and for electrically connecting the nanopore sensor device to the main body.

The EWOD sample preparation device, for the preparation of the prepared sample for testing using the nanopore sensor device may be any suitable and desired device for preparing the prepared sample. The EWOD sample preparation device may be arranged to perform any suitable and desired operations on the raw sample, for preparing it for testing using the nanopore sensor device. This includes interacting (in some instances one or more droplets of) the raw sample with one or more reagents in the interior volume.

In some embodiments, the EWOD sample preparation device is arranged to do one or more (and in some instances all) of: manipulating or manoeuvring (in some instances one or more droplets of) the raw sample within the interior volume (from the inlet to the outlet (i.e. through the interior volume)), mixing (in some instances one or more droplets of) the raw or prepared sample with one or more of the reagents and/or (in some instances one or more other droplets of) the raw or prepared sample, separating (in some instances one or more droplets of) the raw or prepared sample into two or more portions (i.e. smaller droplets) of the raw or prepared sample, subjecting (in some instances one or more droplets of) the raw or prepared sample to one or more interrogation processes, and incubating (in some instances one or more droplets of) the raw or prepared sample in the interior volume.

The EWOD sample preparation device may be arranged to perform similar operations on the one or more reagents. Thus, in some embodiments, the sample preparation device is arranged to do one or more (and in some instances all) of: manipulating (in some instances one or more droplets of) the one or more reagents within the interior volume, mixing (in some instances one or more droplets of) the one or more reagents with (in some instances one or more droplets of) one or more of the other reagents and/or (in some instances one or more other droplets of) the sample, separating (in some instances one or more droplets of) the one or more reagents into two or more portions (i.e. smaller droplets), subjecting (in some instances one or more droplets of) the one or more reagents to one or more interrogation processes, and incubating (in some instances one or more droplets of) the one or more reagents in the interior volume.

It will be appreciated that the EWOD sample preparation device may be arranged to perform these operations on droplet(s) of the raw or prepared sample and/or droplet(s) of the reagent(s) at any suitable and desired point in the process of sample preparation. Thus, the operations may be performed on any suitable and desired state of droplet(s) of the raw or prepared sample, droplet(s) of the reagent(s), mixtures thereof and/or reagents thereof.

The EWOD sample preparation device may be arranged to manipulate and or manoeuvre the sample (within the interior volume) and/or the one or more reagents, mix one or more droplets of the raw or prepared sample and/or the reagent(s) with other droplets of the sample and/or the reagent(s)), and separate one or more droplets of the raw or prepared sample and/or the reagent(s) into two or more portions into smaller droplets of the sample and/or the reagent(s), in any suitable and desired way.

In some embodiments, the EWOD sample preparation device comprises an array of actuation electrodes arranged to do one or more (and in some instances all of): manipulate or manoeuvring one or more droplets of the raw or prepared sample within the interior volume (i.e. from the inlet to the outlet) and/or one or more droplets of the one or more reagents within the interior volume, mix one or more droplets of the raw or prepared sample and/or the reagent(s) (with other droplets of the raw or prepared sample and/or the reagent(s)), and separate one or more droplets of the raw or prepared sample and/or the reagent(s) into two or more portions (i.e. smaller droplets) of the sample and/or the reagent(s).

Preferably the array of actuation electrodes is arranged to manipulate or manoeuvre one or more droplets of the raw or prepared sample and/or the one or more reagents along a (or any) particular predetermined path or paths, respectively within (or through) the interior volume. This may allow the droplet(s) of the raw or prepared sample and/or the reagents(s) to be moved to different locations within the interior volume, e.g. where the raw or prepared sample and/or the reagent(s) may be subject to one or more different process (e.g. being mixed, separated, subjected to interrogation process(es), incubated).

The particular path(s) may be programmed by software that is executed by the EWOD sample preparation device. Thus, in some embodiments, the modular instrument comprises a controller arranged to control the EWOD sample preparation device to do one or more (or all) of: manipulating one or more droplets of the raw or prepared sample and/or the reagent(s), mixing one or more droplets of the raw or prepared sample and/or the reagent(s) (with other (for example droplets) of the raw or prepared sample and/or the reagent(s)), separating one or more droplets of the raw or prepared sample and/or the reagent(s) into two or more portions (for instance smaller droplets) of the raw or prepared sample and/or the reagent(s), subjecting one or more droplets of the raw or prepared sample and/or the reagent(s) to one or more interrogation processes, and incubating one or more droplets of the raw or prepared sample and/or the reagent(s) in the interior volume.

In some embodiments, the EWOD sample preparation device comprises the controller. This may help the controller to be integrated with the components it is controlling. In some embodiments, the main body of the modular instrument comprises the controller. This may allow the functions of the main body and the EWOD sample preparation device to be separated, so that they can be designed and manufactured specifically for these separate functions.

Thus, the EWOD sample preparation device may be arranged to perform one or more (i.e. particular and/or predetermined) preparation protocols which may be programmed by software on the raw or prepared sample and/or the reagent(s).

The EWOD sample preparation device is an electro-wetting on dielectric (EWOD) device, an example of which is disclosed in WO 2019/126715, which is herein incorporated by reference in its entirety.

The (in some instance the EWOD sample preparation device of) the modular instrument may be arranged to subject one or more droplets of the raw or prepared sample and/or the reagent(s) to one or more interrogation processes, and/or incubate one or more droplets of the raw or prepared sample and/or the reagent(s) in the interior volume, in any suitable and desired way. These processes may form part of the protocol being performed by the EWOD sample preparation device.

In some embodiments, the (main body or the EWOD sample preparation device of the) modular instrument comprises a bubble sensor arranged to detect bubbles in the interior volume.

In some embodiments, the (main body or the EWOD sample preparation device of the) modular instrument comprises a heater in thermal communication with the interior volume arranged to heat one or more droplets of the raw or prepared sample and/or the reagent(s) in the interior volume. Thus, the heater may be used to incubate one or more droplets of the raw or prepared sample and/or the reagent(s) in the interior volume.

In some embodiments, the (main body or the EWOD sample preparation device of the) modular instrument comprises a heat sink in thermal communication with the interior volume arranged to dissipate heat from one or more droplets of the raw or prepared sample and/or the reagent(s) in the interior volume.

In some embodiments, the (main body or the EWOD sample preparation device of the) modular instrument comprises a thermoelectric device (e.g. a Peltier element) (e.g. in thermal communication with the interior volume) arranged to cool or heat one or more droplets of the raw or prepared sample and/or the reagent(s) in the interior volume.

In some embodiments, the (main body or the EWOD sample preparation device of the) modular instrument comprises an optical sensor arranged to optically interrogate one or more droplets of the raw or prepared sample and/or the reagent(s) in the interior volume. This may be used to (in examples spectroscopically) identify the sample and/or the reagent(s) in the interior volume, e.g. by conducting a polymerase chain reaction (PCR) test using fluorescence. Thus, the reagent(s) and/or the other components for heating and/or cooling may also be involved in the PCR test, for preparing the raw or prepared sample for the optical sensor.

The interior volume of the EWOD sample preparation device may be provided in any suitable and desired way. Preferably, the interior volume is arranged to contain (i.e. the interior volume contains) a fluid medium. Thus, preferably, the raw or prepared sample and the one or more reagents are arranged to be input into the fluid medium in the interior volume.

The fluid medium may comprise any suitable and desired fluid. The type of fluid may depend on the nature of the raw or prepared sample and/or the reagent(s). In some embodiments, the raw or prepared sample and/or the reagent(s) may comprise polar fluid(s), in this instance the fluid medium comprises an apolar fluid, for example an oil. In some embodiments the raw or prepared sample and/or the reagent(s) may comprise apolar fluid(s)), in this instance the fluid medium comprises a polar fluid, for example a polar solvent, more specifically it may be a polar organic solvent, such as an alcohol.

In some embodiments, the interior volume is removable from the rest of the EWOD sample preparation device. In some embodiments, the interior volume comprises a cartridge. Providing a removable volume may allow the interior volume to be cleaned, flushed and/or washed with, for example, a nuclease, buffer and/or oil, so that it may be reused, and/or may allow the EWOD sample preparation device to be used with different types of interchangeable volumes.

In some embodiments, the EWOD sample preparation device comprises a control body. The interior volume (in examples the cartridge) may be releasably attached to the control body. The control body may thus include one or more (and in some instances all) of: the array of actuation electrodes, the controller, the bubble sensor, the heater, the heat sink, the thermoelectric device, and the optical sensor.

In some embodiments, the control body may be may be integral to (housed in) the EWOD sample preparation device. This may simplify manufacture of the instrument.

In some embodiments, the control body and the (rest of the) EWOD sample preparation device are physically connectable to and separable from each other. This may help to separate the functions of the control body (for example the electrical functions) and the (rest of the) EWOD sample preparation device (for example the sample preparation functions) from each other. Preferably the control body and the (rest of the) EWOD sample preparation device are electrically connectable to and separable from each other. This may allow the control body to control the function(s) of the EWOD sample preparation device.

Similarly, preferably the main body and the control body are physically connectable to and separable from each other. Preferably the main body and the control body are electrically connectable to and separable from each other.

In some embodiments, the main body of the modular instrument comprises a control body. For example, the control body may be integral to (housed in) the main body. Thus, in some embodiments the main body comprises one or more (and in some instances all) of: the controller, the bubble sensor, the heater, the heat sink, the thermoelectric device, and the optical sensor. Preferably the main body is physically connectable to and separable from the EWOD sample preparation device in a way such that these components (as appropriate) may perform their functions on the raw or prepared sample in the interior volume.

It will be appreciated that one or both of the main body and the sample preparation device may comprise a controller, and the main body or the sample preparation device may comprise the bubble sensor, the heater, the heat sink, the thermoelectric device, and the optical sensor, in any suitable and desired combination.

The sample inlet of the interior volume may be arranged in any suitable and desired way, to allow a raw sample to be inserted into the interior volume. In some embodiments, the sample inlet comprises a raw sample inlet aperture arranged to receive one or more droplets of the raw sample. In some embodiments, the inlet (or aperture) is arranged to receive a pipette, for inputting a raw sample into the interior volume. In some embodiments, the inlet (or aperture) is arranged to receive a swab, for inputting a raw sample into the interior volume. In some embodiments, the inlet (or aperture) is arranged to receive a fluid container, for inputting a raw sample into the interior volume.

In some embodiments, the inlet comprises a tube (e.g. a drip (e.g. intravenous) line), for inputting (for example pumping) a raw sample into the interior volume.

In some embodiments, the inlet comprises a lancet, for inputting a raw sample into the interior volume. This allows, for example, a raw blood sample to be obtained directly from a user, without having to obtain the raw sample separately and then input it into the raw sample inlet of the interior volume.

In some embodiments, the raw sample may be input into the interior volume (via the inlet) after raw sample has been obtained from a user and/or has been subject to one or more preparation and/or refining processes. Thus, the present invention also extends to a kit of parts comprising the modular instrument described herein and a raw sample collection device for collecting a raw sample. Preferably, the raw sample collection device is arranged to input the collected raw sample into the interior volume through the raw sample inlet.

The raw sample collection device may comprise any suitable and desired device for collecting a raw sample. For example, the raw sample collection device may comprise a cannula, a lancet, a swab, a pipette, a syringe and/or a tube. The raw sample collection device may comprise a container, e.g. for storing the raw sample and/or transferring the raw sample to the sample inlet.

In some embodiments, the raw sample collection device comprises a container that contains one or more reagents for altering the raw sample for input into the interior volume (via the raw sample inlet). The one or more reagents may be arranged to alter the sample in any suitable and desired way, for example to react with the raw sample or to preserve the raw sample before it is input into the interior volume through the raw sample inlet. The container may, for example, form part of, or be connected to, a cannula, a lancet, a swab, a pipette, a syringe and/or a tube.

The interior volume of the EWOD sample preparation device comprises a plurality of reagent inlets for inputting the one or more reagents into the interior volume. The reagent inlets may be arranged in any suitable and desired way for inputting the one or more reagents into the interior volume.

In some embodiments, the reagent inlets comprise (in examples respective) reagent inlet apertures arranged to receive one or more droplets of the one or more reagents. In some embodiments, the (and in some examples each) inlet (or aperture) is arranged to receive a pipette, for inputting a reagent into the interior volume.

In some embodiments, the reagent inlets comprise (in examples respective) reagent inlet chambers or reservoirs arranged to store one or more droplets of the one or more reagents. This may allow the interior volume to store the reagent(s) before the reagents are used in the interior volume. For example, the reagent(s) may be pre-loaded in the interior volume. The reagent(s) may, for example, be stored in oil.

In some embodiments, the interior volume comprises a blister pack comprising the reagent inlet chambers. Preferably the blister pack is arranged to be actuated (for example to break a frangible membrane(s), to release the one or more reagents into the desired reagent inlet chambers of the interior volume.

The present invention also extends to a kit of parts comprising the modular instrument described herein and a blister pack or reagent cartridge module containing one or more reagents for use with the sample preparation device. Advantageously the blister pack or reagent cartridge module may be stored separately from the device, for example in a refrigerator and fluidically connected to the sample preparation device in use. Preferably the blister pack is arranged to be used to input the one or more reagents into the plurality of reagent inlets. For example, the plurality of reagent chambers of the blister pack may be pressed onto the plurality of reagent inlets. Preferably the action of pressing the blister pack onto the plurality of reagent inlets acts (for example to break a frangible membrane(s) to release the one or more reagents into the reagent inlet chambers of the interior volume.

The one or more reagents may comprise any suitable and desired reagents for preparing the sample and may comprise reagents for preparing the analyte suitable for nanopore sensing, in examples for the library preparation of polynucleotide fragments. In some embodiments, the one or more reagents comprise one or more (and in some instances all) of: a DNA or RNA motor protein, an adaptor, an extraction reagent, a reverse transcription reagent, a polynucleotide amplification reagent such as a polymerase, a fluorescent reagent (for example for DNA fluorescence), a quantification reagent (for example for RNA quantification), a lysis reagent, a bead, comprising a hybridisation reagent, an enzyme inhibitor, a fluorescent dye for quantification of polynucleotides or protein analytes, a buffer, a soluble electrochemical redox couple, PCR reagents such as primers specific for the analyte of interest, a gene targeting reagent such as Cas9 and a magnetic bead reagent (for example for magnetic bead clean-up or purification).

An adaptor reagent may be used for adding an adaptor to a polynucleotide (such as DNA or RNA) fragment, for connecting to a motor protein. A motor protein reagent may be used for adding a motor protein to a polynucleotide (such as DNA or RNA) fragment (via an adaptor).

Suitable reagents for sample preparation are disclosed for example in WO 2014/135838, WO 2012/164270, WO 2015/110813, WO 2018/060740, WO 2016/059375, WO 2019/227013 and WO 2021/111125, hereby incorporated by reference in their entireties. The sample preparation device may further comprise a filter for removing particulates from the sample such as red blood cells.

The EWOD sample preparation device (or kit of parts) may comprise any suitable and desired number of reagents. For example, the sample preparation device (or kit of parts) may comprise greater than 10 reagents, for example greater than 20 reagents, for example greater than 30 reagents, for example greater than 50 reagents.

The outlet of the EWOD sample preparation device, for outputting the prepared sample from the interior volume, may be provided in any suitable and desired way. In some embodiments, the outlet of the EWOD sample preparation device comprises the outlet of the EWOD sample preparation device. In some embodiments, the outlet of the EWOD sample preparation device is in fluid communication with the outlet of EWOD sample preparation device, for example via the port of the EWOD sample preparation device.

Preferably, the outlet is in fluid communication with (i.e. is arranged to output the prepared sample into) the port of the EWOD sample preparation device (into which the nanopore sensor device may be inserted). Similarly, in some embodiments, the EWOD sample preparation device and/or the nanopore sensor device of the modular instrument are arranged such that the inlet of the nanopore sensor device is put into fluid communication with the outlet and/or port of the EWOD sample preparation device, when the nanopore sensor device is displaced relative to (i.e. inserted into, moved within and/or withdrawn from) the outlet and/or port of the EWOD sample preparation device.

Preferably, the EWOD sample preparation device and/or the nanopore sensor device of the modular instrument are arranged such that the inlet of the nanopore sensor device is put into fluid communication with the outlet of the EWOD sample preparation device, when the nanopore sensor device is displaced relative to (i.e. inserted into, moved within and/or withdrawn from) the outlet and/or port of the EWOD sample preparation device.

The outlet of the EWOD sample preparation device (and/or the outlet of the interior volume) may be adjacent to or project into the outlet and/or port of the EWOD sample preparation device. In some embodiments, the outlet comprises a outlet aperture arranged to output or dispense one or more droplets of the raw or prepared sample.

In some embodiments, the outlet comprises a tube, for outputting a prepared sample from the interior volume. The tube may extend from the outlet of the interior volume and/or the outlet of the EWOD sample preparation device into the outlet and/or port of the EWOD sample preparation device.

The prepared sample may be output from the outlet in any suitable and desired way. For example, the prepared sample may be output passively, e.g. allowed to drip out, from the outlet. In some embodiments, the prepared sample may be output actively. Thus, for example, the outlet may comprise a pump for pumping out a sample (for example through the tube of the outlet) from the interior volume. The pump may comprise a blister, for example a pre-formed blister.

In some embodiments, the outlet comprises a proboscis, e.g. that extends (or may be extendible) from the interior volume. The proboscis may be arranged to retract, when the modular instrument is not being used. The proboscis may be arranged to extend (i.e. to project into the outlet and/or port) during use of the modular instrument.

The prepared sample may be output from the outlet in response to the nanopore sensor device being displaced relative to (i.e. inserted into, moved within and/or withdrawn from) the outlet and/or port of the EWOD sample preparation device. Thus, in some embodiments, the inlet of the nanopore sensor device is arranged to engage with the outlet of the EWOD sample preparation device, for example when the nanopore sensor device is displaced relative to (i.e. inserted into, moved within and/or withdrawn from) the outlet and/or port of the EWOD sample preparation device. In this way, the prepared sample may be transferred from the EWOD sample preparation device to the nanopore sensor device automatically, when the nanopore sensor device is displaced relative to the outlet and/or port.

In some embodiments, the EWOD sample preparation device and/or the nanopore sensor device of the modular instrument are arranged such that the inlet of the nanopore sensor device is aligned with the outlet of the EWOD sample preparation device, for example when the nanopore sensor device is displaced relative to (i.e. inserted into, moved within and/or withdrawn from) the outlet and/or port of the EWOD sample preparation device. In some embodiments, the inlet of the nanopore sensor device is arranged to contact the outlet of the EWOD sample preparation device, e.g. when the nanopore sensor device is displaced relative to (i.e. inserted into, moved within and/or withdrawn from) the outlet and/or port of the EWOD sample preparation device.

The sample may be introduced into the sensing device by capillary force. Any conditioning liquid present in the nanopore sensor needs to be displaced by the sample in order to measure the analyte. This can be done in a number of ways. The sample from the outlet can displace the conditioning fluid by controlling the behaviour of the liquids by Laplace pressure and Poiseuille pressure, such as disclosed by WO 2019/106345.

The sensor device may comprise a fluid reservoir whereby conditioning fluid can be caused to flow downstream of the nanopore sensor into the reservoir. The instrument may, for example, comprise an activation means to activate the removal of conditioning fluid for receipt of the prepared sample. The nanopore sensor may for example comprise a valve or flow barrier which can be electronically activated. The activation means may for example be mechanical whereby it is activated by insertion of the nanopore device into the main body or into the sample preparation device. The sample preparation device may comprise means, such as for example a pump to force liquid sample into the nanopore inlet and to displace the conditioning fluid downstream.

The outlet and/or port of the EWOD sample preparation device, into which the nanopore sensor device is arranged to be inserted, may be arranged in any suitable and desired way. In some embodiments, the outlet and/or port is arranged to receive the nanopore sensor device directly into the outlet and/or port. In some embodiments, the outlet and/or port is arranged to receive the nanopore sensor device in a carrier, for example a case or other module.

Thus, the main body of the modular instrument may comprise a carrier arranged to be inserted into the outlet and/or port, wherein the carrier is arranged to carry the nanopore sensor device. Similarly, the nanopore sensor device may be arranged to be inserted into the carrier, for inserting into the outlet and/or port. The carrier may be part of the EWOD sample preparation device, i.e. the outlet and/or port may comprise the carrier. For example, the carrier may comprise a drawer of the (in some instances the outlet and/or port of the) EWOD sample preparation device, wherein the nanopore sensor device is arranged to be inserted into the drawer.

The nanopore sensor device may be any suitable and desired nanopore sensor device, i.e. for sensing molecular entities, e.g. polynucleotides. The nanopore sensor device may, for example, have a construction as disclosed in WO 2009/077734 or WO 2014/064443, which are herein incorporated by reference in their entireties.

Thus, in preferred embodiments, the nanopore sensor device comprises a nanopore membrane (a membrane comprising a nanopore). Preferably the nanopore membrane is in fluid communication with the inlet of the nanopore sensor device. The nanopore membrane may comprise any suitable and desired type of nanopore membrane, e.g. comprising a solid state nanopore or comprising a chemical or biological nanopore. The nanopore sensor device comprise a plurality of nanopore membranes and/or the (in some instances each) nanopore membrane may comprise a nanopore.

The nanopore sensor device preferably comprises one or more wells for containing a fluid, the wells being in fluid communication with the inlet of the nanopore sensor device. The nanopore sensor device preferably comprises one or more sensor electrodes for detecting an ionic current in the one or more wells or for detecting a fluidic electrical potential in the one or more wells (i.e. across the nanopore membrane).

The nanopore sensor device may be reusable. For example, the. one or more wells of the nanopore sensor device may be arranged to be cleaned (i.e. washed and/or flushed with a nuclease, buffer and/or oil), so that it may be reused.

In some embodiments, the modular instrument comprises a detection (also referred to as a signal processing) circuit connected to the nanopore sensor device. In some embodiments, the controller (of the main body, for example), the controller of the EWOD sample preparation device or the controller of the control body comprises the detection circuit. Thus, in some embodiments, the controller and/or the control body is common to (i.e. is arranged to control) both of the EWOD sample preparation device and the nanopore sensor device.

The main body is electrically connectable to and separable from the EWOD sample preparation device, and the main body is electrically connectable to and separable from the nanopore sensor device. The electrical connections may be provided in any suitable and desired way.

The main body may comprise one or more (in some instances all) of: a (for example USB) port for connection to a (remote) computer, a processor for analysing the signals (for example to determine a polynucleotide sequence), a memory (for example for storing algorithms), a display (for example for displaying an indication of the result of the testing being performed on the prepared sample, a physical port for physically connecting to the EWOD sample preparation device, an electrical port for electrically connecting to the EWOD sample preparation device, a physical port for physically connecting to the nanopore sensor device, an electrical port for electrically connecting to the nanopore sensor device. The physical and electrical ports may be provided as an integral physical and electrical port, in any suitable and desired way.

The main body may be arranged to be electrically connectable to and separable from multiple EWOD sample preparation devices and/or multiple nanopore sensor devices. This may allow multiple different samples to be prepared and/or multiple different tests to be performed on the prepared sample(s).

The nanopore sensor device and the corresponding detection circuit may be arranged to perform any suitable and desired type of nanopore sensing of molecular entities. In some embodiments, the nanopore sensor device and the corresponding detection circuit is arranged to perform nanopore sequencing (i.e. data capture and analysis) of at least part (and in some instances all) of the sequence of the molecular entities in the sample, e.g. at least part (and in some instances all) of the sequence of the polynucleotide fragments in the sample.

In some embodiments, the nanopore sensor device and the corresponding detection circuit of the modular instrument are arranged to perform diagnostic testing of the molecular entities in the sample. For example, the nanopore sensor device and the corresponding detection circuit of the modular instrument may be arranged to determine whether or not the sample comprises or does not comprise a type of molecular entity (or entities). This may be used to confirm that the sample is what is expected to be being tested.

The nanopore sensor device the corresponding detection circuit of the modular instrument may be arranged to determine whether or not the sample contains an analyte of interest, for example a (in some instances modified) fragment of DNA or RNA from a virus. This may be used to confirm if the sample passes a test or not, for example to confirm the presence or absence of a virus in the sample.

The nanopore sensor device and the corresponding detection circuit of the modular instrument may be arranged to perform quality control, for example on a fragment of DNA or RNA, for vaccine manufacturing. This may be used to confirm that the sample contains the correctly manufactured fragment of DNA or RNA.

The nanopore sensor device the corresponding detection circuit of the modular instrument may be arranged to determine whether or not the sample contains a particular polynucleotide sequence, for example in a fragment of DNA or RNA, in the sample. This may be used to identify a particular genetic marker, such as for diabetes or cancer, in the sample.

Such diagnostic testing to identify a (or a portion of a) sequence, or a marker in the prepared sample may be performed by the nanopore sensor device and, the corresponding detection circuit itself; or the modular instrument may comprise or be connected to another data processing system. The modular instrument may comprise executing software arranged to perform the analysis of the data collected by the nanopore sensor device and the corresponding detection circuit by the software performing a particular protocol.

Thus, in some embodiments, the control body of the EWOD sample preparation device of the modular instrument may comprise a wired or wireless data connection to a data processing system (in some examples a computer). Preferably the control body of the EWOD sample preparation device of the modular instrument may be arranged to receive and/or transmit data and/or control signals from and/or to the data processing system.

The control body of the EWOD sample preparation device of the modular instrument may comprise an output. The output may be arranged to output (in examples display) a result of the testing being performed on the sample. The output may comprise an indicator of the result of the testing being performed on the prepared sample. In some instances this may be visible or audible to an end user.

This may comprise a positive and/or negative signal, and/or a quantitative result.

### BRIEF DESCRIPTION OF THE FIGURES

Certain preferred embodiments of the invention will now be described, by way of example only, with reference to the accompanying drawings, in which:

FIG. 1 shows schematically a modular instrument for preparing and testing a sample;

FIG. 2 shows schematically a cross-sectional view of part of the modular instrument shown in FIG. 1;

FIG. 3 shows schematically a cross-sectional view of part of a nanopore sensor device of the modular instrument shown in FIG. 1; and FIG. 4 shows schematically a block diagram of the components of the modular instrument and their interconnections.

### DETAILED DESCRIPTION

Various embodiments of the present invention will now be described in the context of nanopore sensors, which may be used for the preparation and sensing of molecular entities.

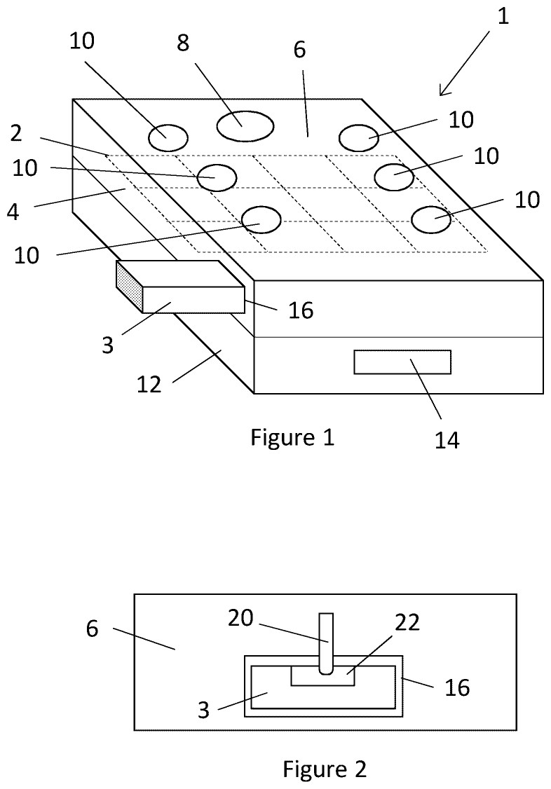

FIG. 1 shows schematically a modular instrument 1 for preparing and testing a prepared sample, in accordance with an embodiment of the present invention. The modular instrument 1 includes an active matrix (AM) arrangement 2, based on thin film electronics including thin-film transistors (TFT), which is used to control the addressing of voltage pulses to an electro-wetting on dielectric (EWOD) device 4. Such an AM-EWOD device is described in WO 2019/126715, WO 2016/110901, WO 2016/166944, WO 2016/111251, WO 2017/038063, WO 2017/047082, WO 2017/047086, WO 2020/183303, WO 2020/260441 and WO 2021/094362.

The AM-EWOD device comprises an interior volume 6 that is filled with a polar or apolar fluid medium, (for example an alcohol or an oil). The interior volume 6 comprises an inlet 8 through which a raw sample is able to be input into the interior volume 6, e.g. using a pipette, drip or swab. The interior volume 6 also comprises multiple reagent inlets 10 through which different reagents are able to be input into the interior volume 6 via, for example, a pipette or from a blister pack of reagent chambers.

The interior volume 6 is in the form of a cartridge that is releasably attached to a control body 12. The control body 12 comprises the actuation electrodes that form the active matrix (AM) arrangement 2. The control body 12 also includes a data connection 14 for connecting the modular instrument 1 to a computer, which may be used to control the modular instrument 1 and/or transfer data to and from the control body 12.

Together, the control body 12 and the AM-EWOD device 4, form an EWOD sample preparation device for the preparation of prepared samples for nanopore sensing. The modular instrument also comprises a nanopore sensor device 3 that is arranged to receive prepared samples from the interior volume 6 of the sample preparation device.

The interior volume 6 comprises a port 16 into which a nanopore sensor device 18 is able to be inserted. The interior volume 6 comprises an outlet that engages with an inlet of the nanopore sensor device 3, when the nanopore sensor device 3 is inserted into the port 16, an example of which will be described with reference to FIG. 2.

FIG. 2 shows schematically a cross-sectional view of the port 16 of the interior volume 6, showing how the outlet from the interior volume 6 interacts with the inlet to the nanopore sensor device 3.

An outlet 20 (e.g. in the form of a tube that is fluidly connected to the interior of the interior volume 6) of the interior volume 6 projects into the port 16. The nanopore sensor device 3 comprises an inlet 22 that is arranged to engage with the outlet 20 of the interior volume, when the nanopore sensor device 3 is inserted into the port 16, in order to transfer a prepared sample (that has been input through the inlet 8 of the interior volume 6 and has been prepared in the interior volume 6) from the interior volume 6 to the nanopore sensor device 3 for testing.

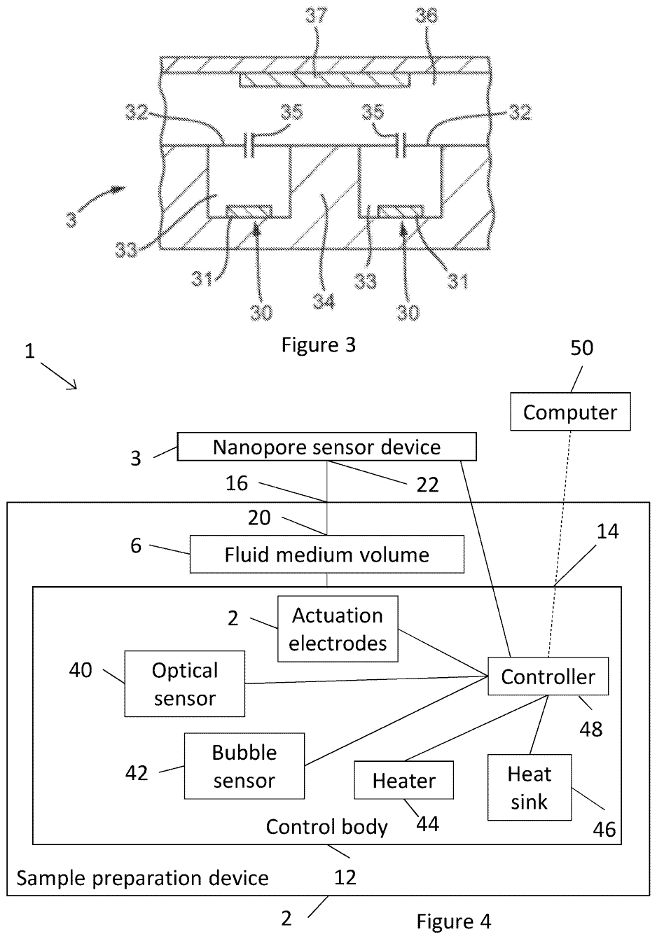

In one example, the nanopore sensor device 3 may have the form shown in FIG. 3, which shows schematically a cross-sectional view of the nanopore sensor device 3. As shown in FIG. 3, the nanopore sensor device 3 comprises an array of sensing elements 30. Each sensing element 30 comprises a membrane 32 supported across a well 33 formed in a support structure 34. A nanopore 35 is inserted in the membrane 32 above each well 33. The membrane 32 may comprise amphiphilic molecules such as a lipid or a polymer.

Each membrane 32 seals the respective well 33 from a sample (“cis”) chamber 36, which extends across the array of sensing elements 30 and is in fluid communication with each nanopore 35. The sample chamber 36 is also in fluid communication with the inlet 22 to the nanopore sensor device 3 (the inlet 22 being shown in FIG. 2).

Each well 33 has a sensor electrode 31 at the base of the (“trans”) well 33. A common electrode 37 is provided in the sample chamber 36 for providing a common reference signal (typically a potential or voltage) to each sensing element 30. In some embodiments, a respective reference electrode for each sensor electrode, e.g. in the well of the sensing element, may be provided.

In use, the sample chamber 36 receives a sample containing molecular entities which interact with the nanopores 35 of the sensing elements 30. In a sensing mode of the nanopore sensor device 3, an ionic current flows from the common electrode 37 to sensor electrodes 31 through the respective nanopores 35. As the molecules transfer from the cis chamber 36 to the trans chamber 33 via the nanopores 35, the path of ions is restricted and thus the ionic current is modulated and is measured by the sensor electrodes 31. This may then be used to characterise and/or identify the molecules. In some embodiments, the nanopore sensor device 3 comprises sensor electrodes that are arranged to measure the fluidic electrical potential in the respective wells across the nanopore membrane.

Two sensing elements 30 are shown in FIG. 3 for clarity, but in general any number of sensing elements 30 may be provided. Typically, a large number of sensing elements 30 may be provided to optimise the data collection rate, for example 256, 1024, 4096 or more sensing elements 30.

The nanopore sensor device 3 may, for example, have a detailed construction as disclosed in WO 2009/077734 or WO 2014/064443, which are herein incorporated by reference in their entireties.

The device comprises an inlet for receiving a liquid sample, the device comprising an upper fluid chamber provided on one side of a nanopore array separating the upper fluid chamber from one or more lower fluid chambers. The one of more lower fluid chambers typically comprise a liquid. Electrodes may be provided in the one or more lower fluid chambers and the upper fluid chamber to provide an electric field across the nanopores. A flow channel may be provided between the inlet and the upper fluid chamber. The analyte is caused to pass through the nanopores under an electric field and electrical signals such as the measurement of ion current flow through the nanopores may be measured to determine the analyte of interest.

Analytes of interest include natural or synthetic polynucleotides such as RNA or DNA, polypeptides and polysaccharides. A conditioning fluid may be provided in the upper fluid chamber to cover the nanopore array and to prevent evaporation of liquid from the one of more lower fluid chambers.

FIG. 4 shows schematically a block diagram of the components of the modular instrument 1 and their interconnections.

The modular instrument 1 includes the EWOD sample preparation device 2 (as shown in FIGS. 1 and 2) and the nanopore sensor device 3 (as shown in FIGS. 1, 2 and 3). The EWOD sample preparation device 2 includes the control body 12 and the fluid medium volume 6, in the form of a cartridge, which is releasably attached to the control body 12.

The EWOD sample preparation device 2 comprises a port 16, into which the nanopore sensor device 3 is able to be inserted, such that the outlet 20 of the fluid medium volume 6 is put into fluid communication with the inlet 22 of the nanopore sensor device 3.

The control body 12 includes an array of actuation electrodes of the active matrix (AM) arrangement 2 arranged in proximity to the fluid medium volume 6, an optical sensor 40 arranged in optical communication with the fluid medium volume 6, a bubble sensor 42 arranged in proximity to the fluid medium volume 6, a heater 44 arranged in thermal communication with the fluid medium volume 6, a heat sink 46 arranged in thermal communication with the fluid medium volume 6, and a controller 48.

The controller 48 is in data communication with an external computer 50, via a data port 14 of the control body 12 (for example a USB connection). The controller 48 is also in communication with each of the actuation electrodes 2, the optical sensor 40, the bubble sensor 42, the heater 44 and the heat sink 46. The controller 48 is arranged to control operation of each of these components, so to control the preparation of the sample in the fluid medium volume 6.

The controller 48 is also in communication with the nanopore sensor device 3, so to control operation of the nanopore sensor device 3 and to receive the signals from the sensor electrodes 31 (as shown in FIG. 3).

Operation of the modular instrument 1, for the preparation of a prepared sample for, and the testing of the prepared sample by, the nanopore sensor device 3 will now be described with reference to FIGS. 1 to 4. The preparation of the sample may, for example, include some of the steps outlined in WO 2019/126715.

To prepare the modular instrument 1 for operation, the fluid medium volume 6 is filled with a fluid medium, e.g. a polar or apolar fluid medium, for example an alcohol or an oil. A droplet of a raw sample to be prepared is input into the fluid medium volume 6 through the raw sample inlet 8 and the reagents for the sample preparation are loaded into the reagent inlets 10.

The reagents may, for example, include reagents for the library preparation of polynucleotide fragments, e.g. one or more (in some instances all) of: a motor protein, an adaptor, an extraction reagent, a reverse transcription reagent, an amplification reagent, a fluorescent reagent, a quantification reagent, and a magnetic bead reagent.

The controller 48, under the control of software being executed on the computer 50, controls the actuation electrodes 2 (for example according to a protocol being run by the software and/or the controller 48) to do one or more (in some instances all) of: manipulate droplets of the raw or prepared sample within the interior volume along a predetermined path from the inlet to the outlet, manipulate droplets of the reagents within the interior volume, mix droplets of the raw or prepared sample and/or the reagents with other such droplets, and separate droplets of the raw or prepared sample and/or the reagents into smaller droplets.

The controller 48 also controls the optical sensor 40, the bubble sensor 42, the heater 44 and the heat sink 46, so that droplets of the raw or prepared sample and/or the reagents in the proximity of these components can be interrogated and/or exposed to the thermal effects of the components. Thus, the optical sensor 40 is operated (for example according to the protocol) to optically interrogate droplets of the raw or prepared sample and/or the reagents in the interior volume, e.g. to spectroscopically identify the raw or prepared sample and/or the reagents in the interior volume, e.g. by conducting a polymerase chain reaction (PCR) test, e.g. using fluorescence.

The bubble sensor 42 is operated (for example according to the protocol) to detect bubbles in the interior volume.

The heater 44 is operated (for example according to the protocol) to heat droplets of the sample and/or the reagents in the interior volume, e.g. to incubate the sample and/or the reagents.

The heat sink 46 is operated (for example according to the protocol) to dissipate heat from droplets of the sample and/or the reagents in the interior volume.

Having prepared the prepared sample, for example according to the protocol, by manipulating the droplet of the raw or prepared sample and the reagents within the fluid medium volume 6, the droplet of the sample is moved to the outlet 20 of the fluid medium volume 6. The nanopore sensor device 3 is then inserted into the port 16 of the EWOD sample preparation device 2 and this action engages the inlet 22 of the nanopore sensor device 3 with the outlet 20 of the fluid medium volume 6.

With the prepared sample now received by the inlet 22 of the nanopore sensor device 3, the nanopore sensor device 3 is operated (for example according to a protocol controlled by the controller 48 to control the detection circuit of the nanopore sensor device 3) to perform nanopore sequencing (for example data capture and analysis) of at least part (and in some instances. All) of the sequence of the molecules (i.e. polynucleotide fragments) in the sample.

The nanopore sequencing may be used for sequencing of polymers such as polynucleotide fragments and/or for performing diagnostic testing of the sample.

1. A modular instrument for preparing and sensing an analyte in a prepared sample, the modular instrument comprising:
a main body;
an EWOD sample preparation device for the preparation of the prepared sample for use with a nanopore sensor device; and
a nanopore sensor device for sensing the analyte;
wherein the main body is electrically connectable to and separable from both the EWOD sample preparation device and the nanopore sensor device;
wherein the main body, the EWOD sample preparation device and nanopore sensor are physically connectable to and separable from each other; and
wherein the EWOD sample preparation device comprises:
an interior volume for interacting a raw sample with one or more reagents;
a sample inlet for inputting the raw sample into the interior volume;
a plurality of reagent inlets for inputting the one or more reagents into the interior volume; and
an outlet for outputting the prepared sample into an inlet of the nanopore sensor device for sensing of the analyte, wherein the outlet of the EWOD sample preparation device is fluidically connectable to the inlet of the nanopore device.

2. The modular instrument as claimed in claim 1, wherein the EWOD sample preparation device comprises an array of actuation electrodes arranged to do one or more of:
manipulate or manoeuvre the raw or prepared sample and/or the one or more reagents;
mix the raw or prepared sample and/or the one or more reagents with other of the raw or prepared sample and/or the one or more reagents; and
separate the raw or prepared sample and/or the one or more reagents into two or more portions of the raw or prepared sample and/or the one or more reagents.

3. The modular instrument as claimed in claim 2, wherein the array of actuation electrodes is arranged to manipulate or manoeuvre the raw or prepared sample and/or the one or more reagents along a particular path within the interior volume.

4. The modular instrument as claimed in claim 1, 2 or 3, wherein the EWOD sample preparation device comprises a bubble sensor arranged to detect bubbles in the interior volume.

5. The modular instrument as claimed in any one of the preceding claims, wherein the EWOD sample preparation device comprises a heater arranged to heat the raw or prepared sample and/or the one or more reagents in the interior volume.

6. The modular instrument as claimed in any one of the preceding claims, wherein the EWOD sample preparation device comprises a heat sink arranged to dissipate heat from the raw or prepared sample and/or the one or more reagents in the interior volume.

7. The modular instrument as claimed in any one of the preceding claims, wherein the EWOD sample preparation device comprises a thermoelectric device arranged to cool or heat the raw or prepared sample and/or the one or more reagents in the interior volume.

8. The modular instrument as claimed in any one of the preceding claims, wherein the main body comprises an optical sensor arranged to optically interrogate the raw or prepared sample and/or the one or more reagents in the interior volume.

9. The modular instrument as claimed in any one of the preceding claims, wherein the interior volume contains a fluid medium, wherein the fluid medium comprises an apolar fluid or a polar fluid.

10. The modular instrument as claimed in any one of the preceding claims, wherein the interior volume is removable.

11. The modular instrument as claimed in claim 10, wherein the EWOD sample preparation device comprises a control body, wherein the interior volume is releasably attached to the control body.

12. The modular instrument as claimed in any one of the preceding claims, wherein the raw sample inlet is arranged to receive a pipette, a swab or a fluid container.

13. The modular instrument as claimed in any one of the preceding claims, wherein the raw sample inlet comprises a tube or a lancet for inputting a sample into the interior volume.

14. The modular instrument as claimed in any one of the preceding claims, wherein the reagent inlets comprise reagent inlet chambers arranged to store the one or more reagents.

15. The modular instrument as claimed in claim 14, wherein the interior volume comprises a blister pack comprising the reagent inlet chambers, wherein the blister pack is arranged to be actuated to release the one or more reagents into the interior volume.

16. The modular instrument as claimed in any one of the preceding claims, wherein the one or more reagents comprise one or more of: a motor protein, an adaptor, an extraction reagent, a reverse transcription reagent, an amplification reagent, a fluorescent reagent, a quantification reagent, a PCR reagent, a gene targeting reagent and a magnetic bead reagent.

17. The modular instrument as claimed in any one of the preceding claims, wherein EWOD sample preparation device comprises a port for inserting the nanopore sensor device, wherein the interior volume comprises an outlet, and wherein the outlet of the interior volume is in fluid communication with the port of the EWOD sample preparation device.

18. The modular instrument as claimed in any one of the preceding claims, wherein the modular instrument is arranged such that the inlet of the nanopore sensor device is put into fluid communication with the outlet of the EWOD sample preparation device, when the nanopore sensor device is displaced relative to the outlet of the EWOD sample preparation device.

19. The modular instrument as claimed in any one of the preceding claims, wherein the outlet comprises a tube or a proboscis, for outputting the prepared sample from the interior volume.

20. The modular instrument as claimed in any one of the preceding claims, wherein the nanopore sensor device is arranged to engage with the outlet of the EWOD sample preparation device, when the nanopore sensor device is displaced relative to the outlet of the EWOD sample preparation device.

21. The modular instrument as claimed in any one of the preceding claims, wherein the system is arranged such that the inlet of the nanopore sensor device is aligned with the outlet of the EWOD sample preparation device, when the nanopore sensor device is displaced relative to the outlet of the EWOD sample preparation device.

22. The modular instrument as claimed in any one of the preceding claims, wherein the nanopore sensor device comprises a nanopore membrane.

23. The modular instrument as claimed in claim 22, wherein the nanopore membrane is in fluid communication with the inlet of the nanopore sensor device.

24. The modular instrument as claimed in any one of the preceding claims, wherein the nanopore sensor device is arranged to perform nanopore sequencing of at least part of the sequence of the molecular entities in the sample.

25. The modular instrument as claimed in any one of the preceding claims, wherein the system is arranged to perform diagnostic testing of the molecular entities in the sample.
